SEQUENCE LISTING (3 sequences)

SEQ ID NO: 1 - DNA, 4418 bp, Artificial Sequence
  Description of Artificial Sequence Synthetic construct
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctctagcccgggcggatccgaattcgcatgcgtcgactcgaggactacaaggatgacgatgacaaggattacaaagacgacgatgataaggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccatggtgagcaagcagatcctgaagaacaccggcctgcaggagatcatgagcttcaaggtgaacctggagggcgtggtgaacaaccacgtgttcaccatggagggctgcggcaagggcaacatcctgttcggcaaccagctggtgcagatccgcgtgaccaagggcgcccccctgcccttcgccttcgacatcctgagccccgccttccagtacggcaaccgcaccttcaccaagtaccccgaggacatcagcgacttcttcatccagagcttccccgccggcttcgtgtacgagcgcaccctgcgctacgaggacggcggcctggtggagatccgcagcgacatcaacctgatcgaggagatgttcgtgtaccgcgtggagtacaagggccgcaacttccccaacgacggccccgtgatgaagaagaccatcaccggcctgcagcccagcttcgaggtggtgtacatgaacgacggcgtgctggtgggccaggtgatcctggtgtaccgcctgaacagcggcaagttctacagctgccacatgcgcaccctgatgaagagcaagggcgtggtgaaggacttccccgagtaccacttcatccagcaccgcctggagaagacctacgtggaggacggcggcttcgtggagcagcacgagaccgccatcgcccagctgaccagcctgggcaagcccctgggcagcctgcacgagtgggtgtaatagggtaccaggtaagtgtacccaattcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgtatggggattggtggcgacgactcctggagcccgtcagtatcggcggaattccagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 4554 bp, Artificial Sequence
  Description of Artificial Sequence Synthetic construct
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcacccgggcggatccgaattcgcatgcgtcgactcgaggactacaaggatgacgatgacaaggattacaaagacgacgatgataaggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccatggtgagcaagcagatcctgaagaacaccggcctgcaggagatcatgagcttcaaggtgaacctggagggcgtggtgaacaaccacgtgttcaccatggagggctgcggcaagggcaacatcctgttcggcaaccagctggtgcagatccgcgtgaccaagggcgcccccctgcccttcgccttcgacatcctgagccccgccttccagtacggcaaccgcaccttcaccaagtaccccgaggacatcagcgacttcttcatccagagcttccccgccggcttcgtgtacgagcgcaccctgcgctacgaggacggcggcctggtggagatccgcagcgacatcaacctgatcgaggagatgttcgtgtaccgcgtggagtacaagggccgcaacttccccaacgacggccccgtgatgaagaagaccatcaccggcctgcagcccagcttcgaggtggtgtacatgaacgacggcgtgctggtgggccaggtgatcctggtgtaccgcctgaacagcggcaagttctacagctgccacatgcgcaccctgatgaagagcaagggcgtggtgaaggacttccccgagtaccacttcatccagcaccgcctggagaagacctacgtggaggacggcggcttcgtggagcagcacgagaccgccatcgcccagctgaccagcctgggcaagcccctgggcagcctgcacgagtgggtgtaatagggtaccaggtaagtgtacccaattcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgtatggggattggtggcgacgactcctggagcccgtcagtatcggcggaattccagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 3 - DNA, 7719 bp, Artificial Sequence
  Description of Artificial Sequence Synthetic construct
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcacccgggcggatccgccgccgccatgaaagtgttccgcaattccgcaaaaaagaagaggaaggtagaagaccccaaggactttccttcagaattgctaagttttttgagtccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcagggtcgactcgaggactacaaggatgacgatgacaaggattacaaagacgacgatgataaggactataaggatgatgacgacaaataatagcaattcctcgacgactgcatagggttacccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacaaccatggtgagcaagcagatcctgaagaacaccggcctgcaggagatcatgagcttcaaggtgaacctggagggcgtggtgaacaaccacgtgttcaccatggagggctgcggcaagggcaacatcctgttcggcaaccagctggtgcagatccgcgtgaccaagggcgcccccctgcccttcgccttcgacatcctgagccccgccttccagtacggcaaccgcaccttcaccaagtaccccgaggacatcagcgacttcttcatccagagcttccccgccggcttcgtgtacgagcgcaccctgcgctacgaggacggcggcctggtggagatccgcagcgacatcaacctgatcgaggagatgttcgtgtaccgcgtggagtacaagggccgcaacttccccaacgacggccccgtgatgaagaagaccatcaccggcctgcagcccagcttcgaggtggtgtacatgaacgacggcgtgctggtgggccaggtgatcctggtgtaccgcctgaacagcggcaagttctacagctgccacatgcgcaccctgatgaagagcaagggcgtggtgaaggacttccccgagtaccacttcatccagcaccgcctggagaagacctacgtggaggacggcggcttcgtggagcagcacgagaccgccatcgcccagctgaccagcctgggcaagcccctgggcagcctgcacgagtgggtgtaatagggtaccaggtaagtgtacccaattcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgtatggggattggtggcgacgactcctggagcccgtcagtatcggcggaattccagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

(19) United States
(12) Patent Application Publication
(10) Pub. No.: US 2007/0134767 A1
(43) Pub. Date: Jun. 14, 2007

(54) Hiv-dependent expression constructs and uses therefor

(75) Inventors: Yuntao Wu, Manassas, VA (US); Jon W. Marsh, Bethesda, MD (US)

Correspondence Address: EDWARDS ANGELL PALMER & DODGE LLP;(CLIENT REFERENCE NO. 47992), PO BOX 55874, BOSTON, MA, 02205, US

(73) Assignee: Government of the US, as represented by the Secretary, Department of Health and Human Services, Rockville, MD (US)

(21) Appl. No.: 10/574,031

(22) PCT Filed: Sep. 28, 2004
(86) PCT No.: PCT/US04/31967

§ 371 Date: Jun. 20, 2006

Related U.S. Application Data
(60) Provisional application No. 60/507,034, filed on Sep. 28, 2003.

(51) Int. Cl. C12Q 1/68; C12P 19/34

(52) U.S. Cl. 435/91.1; 435/6

(57) ABSTRACT

The present invention provides nucleic acid molecules comprising expressible sequences, including reporter genes and therapeutic genes, whose expression is dependent on the presence of both HIV Tat and Rev proteins. Further provided are methods for detecting HIV, method for identifying compounds that can inhibit HIV infection and/or gene expression, methods for killing HIV-infected cells, and methods of treating HIV-infected subjects.

### RELATED APPLICATIONS

This applications claim the benefit of U.S. Provisional Application Ser. No. 60/507,034, filed on Sep. 28, 2003, the entire contents of which are hereby incorporated by this reference.

### BACKGROUND OF THE INVENTION

1. Field of the Invention

The present invention features nucleic acid molecules comprising expressible sequences, including reporter genes and therapeutic genes, whose expression is dependent on the presence of both HIV Tat and Rev proteins. Further featured are methods for detecting HIV, methods for identifying compounds that can inhibit HIV infection and/or gene expression, methods for killing HIV-infected cells, and methods of treating HIV-infected subjects.

2. Background

Acquired Immune Deficiency Syndrome (AIDS) caused by Human Immunodeficiency Virus (HIV) infection is a leading cause of illness and death in the United States and worldwide. Treatment of AIDS with available drugs is frequently ineffective due to either endogenous or acquired resistance. Because early diagnosis of HIV infection may be critical for the success of existing treatment regimens, the development of more sensitive and more accurate diagnostic tests for HIV infection is extremely important.

In the Unites States, more than 688,000 cases of AIDS have been reported since 1981, and the rate of new infections remains at an unacceptably high level of 40,000 per year. Half of all newly infected individuals are people under 25, and minority populations are disproportionately affected: Worldwide, approximately one in every 100 adults aged 15 to 49 is infected with HIV. There were an estimated 5.6 million new HIV infections worldwide in 1999, or approximately 15,000 infections daily. More than 95% of these new infections were in developing countries. By the year 2003, almost 40 million people were estimated to be infected with HIV worldwide (see NIAID website).

The development of methods which will aid the diagnosis of HIV infection, provide a means to kill HIV infected cells, and allow the identification of new therapeutic agents for treating HIV will be of tremendous importance in AIDS treatment. Accordingly, there is an acute need in the art for such methods.

Retroviruses, such as HIV, undergo reverse transcription to form double stranded DNA, which is then integrated into the host chromatin. The integrated provirus transcribes new genomic and messenger RNAs for virion production. HIV possesses the typical three retroviral genes, gag, pol, and env, on a 9 kilo-base genome. The viral genome also encodes 6. accessory or regulatory genes. The expression of this unusually high number of gene products is accomplished by use of multiple reading frames and multiple splicing sites.

Transcription from the provirus is regulated by the activity of the HIV promoter, the long terminal repeat (LTR) found at the 5′ end of the DNA. The LTR possesses binding sites for numerous cellular transcription factors, including Sp1, NFkB, AP-1, and NF-AT (Garcia, J. A. et al. (1987) EMBO J. 6:3761-70; Kawakami, K. et al. (1988) Proc. Natl. Acad. Sci. USA 85:4700-4; Leonard, J. et al. (1989) J. Virol. 63:4919-24; Li, C. et al. (1991) Proc. Natl. Ac ad. Sci. USA 88:7739-43; Nabel, G. and Baltimore, D. (1987) Nature 326:711-3 [published erratum appears in Nature (1990) 344(6262):178]; Ross, E. K. et al. (I 991) J. Virol. 65:4350-8). Given that these factors are responsible for T cell activity, it is not surprising that T cell activation promotes viral expression (Siekevitz, M. et al. (1987) Science 238:1575-8 [published erratum appears in Science (1988) 239(4839):451]; Stevenson, M. et al. (1990) EMBO J. 9:1.551-60; Tong-Starksen, S. E. et al. (1987) Proc. Natl. Acad. Sci. USA 84:6845-9). In the absence of premature termination, expression from the provirus results in the generation of a single “full length” RNA species. This non-spliced transcript serves as messenger for several HIV structural proteins (gag-pol genes), as well as the RNA genome that is incorporated into newly synthesized HIV particles. There are events in normal HIV infection, however, that precede the accumulation of new genomic RNA. Common for host and retroviral gene expression, co-transcriptional association of the forming message with an assortment of proteins—including splicing enzymes—results in the removal of introns and efficient delivery of the mature message to the cytosol. The full-length HIV transcript also contains a variety of splicing donors and acceptor sites. This feature of HIV permits the encoding of various proteins in overlapping genes (within the same segment of DNA), and permits a temporal separation of gene expression. Through varied use and non-use of splicing sites, the single RNA generated from the integrated DNA can yield nearly forty different transcripts that encode a total of nine different proteins (Purcell, D. F. and Martin, M. A. (1993) J. Virol. 67:6365-78). In the infected cell, the earliest RNA generated becomes fully spliced by the cellular splicing machinery.

Fully spliced HIV transcripts encode three proteins: negative factor Nef, trans-activator of transcription Tat, and the regulator of viral gene expression Rev. These three gene products are regulatory proteins that affect cellular and viral functions that lead to efficient viral replication, but more specifically, all three can alter the viral transcription output. Tat and Rev associate with regions of newly transcribing HIV RNA. Tat associates co-transcriptionally (along with numerous cellular protein factors, including an RNA polymerase II-modifying kinase) with a 5′ stem-loop structure TAR (Rana, T. M. and Jeang, K. T. (1999) Arch. Biochem. Biophys. 365:175-185). Tat and the associated proteins function by promoting completion of initiated transcriptional activity (processivity or anti-termination). Rev protein is responsible for the conversion from early HIV gene expression to late gene expression in the newly infected cells. Rev mediates the cytosolic delivery of singly and non-spliced message, and thus its expression coordinates the conversion of predominately Nef, Tat, and Rev (products of multiply spliced transcript) to expression of singly and unspliced HIV transcripts, such as those for the structural proteins of the virion (Pollard, V. W. and Malim, M. H. (1998) Annu. Rev. Microbiol. 52:491-532). This occurs through a physical interaction of Rev with unspliced or singly spliced transcripts and with cellular components that are responsible for message export from the nucleus. The RNA region for Rev association, the Rev-responsive element (RRE), is located in the 3′ half of the HIV RNA within the env gene. Multiple copies of Rev assemble on the RRE and a different region of Rev associates with the CRM1 nuclear export protein. This association mediates transport of the transcripts to the cytosol. The association of RNA-free Rev with importin-β in the cytosol results in the return trip of Rev protein to the nucleus.

The presence of Tat or Rev is indicative of HIV infection, and both HIV proteins affect expression from the integrated HIV provirus. As Tat enhances expression from an LTR-driven gene, the LTR coupled to a reporter gene is commonly used to demonstrate the presence of HIV, such as in HIV-indicator cells. Such cells possess an integrated LTR upstream to a reporter, such as β-galactosidase (Kimpton, J. and Emerman, M. (1992) J. Virol. 66:2232-2239; Vodicka, M. A. et al. (1997) Virology 233:193-198), luciferase (Aguilar-Cordova, E. et al. (1994) AIDS Res. Hum. Retroviruses 10:295-301), chloramphenicol acetyltransferase (Ciminale, V. et al. (1990) AIDS Res. Hum. Retroviruses 6:1281-1287; Schwartz, S. et al. (1989) Proc. Natl. Acad. Sci. USA 86:7200-7203), or green fluorescent protein (Gervaix, A. et al. (1997) Proc. Natl. Acad. Sci. USA 94:4653-4658). Indeed, all of the indicator lines listed in the NIH NIAID Research and Reference Reagent Program for HIV and SIV (including those mentioned above) utilize the LTR sensitivity to Tat expression.

However, Tat-dependent indicator cells are not optimal for a number of reasons, including the fact that the HIV LTR is responsive to other cellular factors. This can lead to an undesirable level of background activation. Accordingly, there is a need in the art for more specific methods of testing for HIV infection.

### SUMMARY OF THE INVENTION

The present invention is based, at least in part, on the discovery of novel DNA constructs, referred to herein as “HIV-dependent expression construct”, “HDEC”, or simply “expression construct” nucleic acid molecules, which comprise an expressible sequence whose expression is dependent on the presence of both HIV Tat and Rev proteins. HIV Tat regulates transcription of the expressible sequence mRNA. However, because the expressible sequence is contained, at least in part, within an intron, it is spliced out by the cellular splicing machinery unless Rev is present. Accordingly, these novel expression constructs are capable of detecting HIV infection and/or gene expression with both specificity and sensitivity. They may also be useful in screening assays for compounds capable of inhibiting HIV infection and/or gene expression. They may also be useful for killing HIV-infected cells through the use of cytotoxic expressible sequences.

Accordingly, in one embodiment, the invention provides isolated nucleicacid molecules comprising: a promoter, wherein the activity of the promoter is dependent on the presence of the human immunodeficiency virus (HIV) Tat protein (e.g., the HIV 5′ LTR); at least one splice donor site (e.g., the HIV D1 splice donor site) and at least one splice acceptor site (e.g., the HIV A7 splice donor site); an expressible sequence which is not a wild-type HIV sequence, wherein at least part of the reporter gene is located in an intron between the splice acceptor site and the splice donor site; and a Rev Responsive Element (RRE) from the human immunodeficiency virus. In another preferred embodiment, the nucleic acid molecules of the invention further comprise the human HIV 3′ LTR. In one embodiment, the splice acceptor site is contained within the RRE.

In another embodiment, the nucleic acid molecules of the invention further comprise at least a second splice donor site (e.g., the HIV D4 splice acceptor site) and at least a second splice acceptor site (e.g., the HIV A5 splice acceptor site). In still another embodiment, the nucleic acid molecules of the invention comprise a psi (φ) site, and/or an internal ribosome entry site (IRES).

In one embodiment, the expressible sequence comprises a reporter gene, for example, a reporter gene that encodes a fluorescent protein (e.g., green fluorescent protein (GFP), enhanced green fluorescent protein (EGFP), red fluorescent protein (RFP), yellow fluorescent protein (YFP), enhanced yellow fluorescent protein (EYFP), blue fluorescent protein (BFP), or cyan fluorescent protein (CFP)). In another embodiment, the reporter gene encodes luciferase (e.g., firefly luciferase or Renilla luciferase), β-galactosidase, thymidine kinase (TK), or chloramphenicol acetyl transferase (CAT). In anther embodiment, the reporter gene comprises a therapeutic gene (e.g., a cytoxic protein).

In a preferred embodiment, the isolated nucleic acid molecules of the invention include the nucleotide sequence set forth in SEQ ID NO:1, 2, or 3, or the insert contained within the plasmid deposited with the ATCC as Accession No. ______, or a complement thereof. In another embodiment, an isolated nucleic acid molecule of the invention comprises a nucleotide sequence which is at least about 50%, 55%, 60%, 65%, 70%, 75%, 80%, 85%, 90%, 91%, 92%, 93%, 94%, 95%, 96%, 97%, 98%, 98.5%, 99%, 99.25%, 99.5%, 99.6%, 99.7%, 99.8%, or 99.9% identical to the nucleotide sequence of SEQ ID NO:1, 2, or 3, or the insert contained within the plasmid deposited with the ATCC as Accession No. ______, wherein expression of the expressible sequence is dependent on the presence of HIV Tat and Rev proteins.

In another embodiment, the invention provides isolated nucleic acid molecules comprising the complement (e.g., a fill complement) of the nucleic acid molecules described herein.

In another embodiment, the invention provides vectors (e.g., plasmids and recombinant retroviruses) and host cells (e.g., T cells, or the host cell deposited with the ATCC as Accession No. ______) containing the nucleic acid molecules of the invention.

In still another embodiment the invention provides a method of determining whether HIV is present in a sample comprising: contacting a host cell containing a nucleic acid molecule of the invention with the sample; culturing the cell for an amount of time sufficient to allow HIV infection and gene expression; and determining whether the expressible sequence is expressed by the cell, wherein expression of the expressible sequence is indicative of the presence of HIV in the sample. In a preferred embodiment, the biological sample is isolated from a subject (e.g., a human subject). In a further preferred embodiment, the biological sample is selected from the group consisting of a biological fluid samiple (e.g., blood, serum, plasma, saliva, urine, stool, semen, vaginal fluid, spinal fluid, lymph, amniotic fluid, tears, nasal secretions, sweat, breast milk, mucus, or interstitial fluid), a tissue sample (e.g., a lymph node sample, a skin sample, or a chorionic villus sample), and a cell sample (e.g., a blood cell sample such as a T cell sample). In a further embodiment, the sample may be purified.

In another embodiment, the invention provides a method of determining whether a cell (e.g., a T cell) is infected with HIV comprising: contacting the cell with the retrovirus containing a nucleic acid molecule of the invention; culturing the cell for an amount of time sufficient to allow HIV gene expression; and determining whether the expressible sequence is expressed by the cell, wherein expression of the expressible sequence is indicative of HIV infection of the cell.

In yet another embodiment, the invention provides a method of determining whether a subject (e.g., a human subject) is infected with HIV comprising contacting the cells of the subject with a retrovirus containing a nucleic acid molecule of the invention, and determining whether the expressible sequence is expressed by the cells, wherein expression of the expressible sequence is indicative of HIV infection.

In still another embodiment, the invention provides a method of killing a cell infected with HIV (e.g., a T cell) comprising contacting a retrovirus containing a nucleic acid molecule of the invention, wherein the retrovirus contains an expressible sequence that encodes a cytotoxic protein. In a preferred embodiment, the cells are contained within a human subject.

In another embodiment, the invention provides a method of treating a subject (erg., a human subject) infected with HIV comprising administering to the subject a retrovirus containing a nucleic acid molecule of the invention, wherein the retrovirus contains an expressible sequence that encodes a cytotoxic protein.

In another embodiment, the invention provides a method of identifying a compound capable of inhibiting HIV infection or gene expression or comprising: contacting a host cell containing a nucleic acid molecule of the invention with a test compound; contacting the cell with HIV; culturing the cell for an amount of time sufficient to allow HIV infection and gene expression; and determining whether the expressible sequence is expressed by the cell, wherein a compound that inhibits expression of the expressible sequence is identified as a compound that is capable of inhibiting HIV infection or gene expression.

In yet another embodiment, the invention provides a method of identifying a compound capable of inhibiting HIV infection or gene expression or comprising: contacting a cell with HIV; contacting the cell with a retrovirus containing a nucleic acid molecule of the invention; contacting the cell with a test compound; culturing the cell for an amount of time sufficient to allow HIV infection and gene expression; and determining whether the expressible sequence is expressed by the cell, wherein a compound that inhibits expression of the expressible sequence is identified as a compound that is capable of inhibiting HIV infection or gene expression.

In still another embodiment of the invention, the invention provides a method of identifying a compound capable of inhibiting HIV infection or gene expression or comprising: contacting a cell infected with HIV with a retrovirus containing a nucleic acid molecule of the invention; contacting the cell with a test compound; culturing the cell for an amount of time sufficient to allow HIV infection and gene expression; and determining whether the expressible sequence is expressed by the cell, wherein a compound that inhibits expression of the expressible sequence is identified as a compound that is capable of inhibiting HIV infection or gene expression.

Other features and advantages of the invention will be apparent from the following detailed description and claims.

### BRIEF DESCRIPTION OF THE DRAWINGS

FIG. 1 depicts the nucleic acid sequence of the HIV-dependent expression construct of SEQ ID NO:1, which contains a GFP reporter gene and a single splice acceptor/splice donor site pair.

FIG. 2 depicts the nucleic acid sequence of the HIV-dependent expression construct of SEQ ID NO:2, which contains a GFP reporter gene and two splice acceptor/splice donor site pairs.

FIGS. 3A-3B depicts the nucleic acid sequence of HIV-dependent expression construct of SEQ ID NO:3, which contains a GFP reporter gene, a β-galactosidase reporter gene, and two splice acceptor/splice donor site pairs.

FIG. 4 depicts a schematic of an exemplary HIV-dependent expression construct containing a single splice acceptor/splice donor site pair. The relative positions of the 5′ LTR, the splice donor site (D1), the expressible sequence (ORF), the Rev responsive element (RRE), the splice acceptor site (A7), and the 3′ LTR are indicated. Also shown are the resulting mRNA transcripts in the absence (spliced) and the presence (unspliced) of Rev.

FIG. 5 depicts a schematic of an exemplary HIV-dependent expression construct containing two splice acceptor/splice donor site pairs. The relative positions of the 5′ LTR, the splice donor sites (D1 and D4), the expressible sequence (ORF), the Rev responsive element (RRE), the splice acceptor sites (A4 and A7), and the 3′ LTR are indicated. Also shown are the resulting mRNA transcripts in the absence (spliced) and the presence (unspliced) of Rev.

FIG. 6 depicts a gel showing the RNA extracted from CEM T cells infected with HIV and with a retrovirus containing the HIV-dependent expression construct (HDEC) of SEQ ID NO:2. Lane 1: control (−HDEC, −HIV); Lane 2: control (+HDEC, −HIV); Lane 3: control (−HDEC, +HIV); Lane 4: +HDEC, +HIV.

FIG. 7 depicts GFP fluorescence of CEM T cells infected with a retrovirus containing the HIV-dependent expression construct (HDEC) of SEQ ID NO:2, with (bottom) or without (top) infection with HIV.

FIG. 8 depicts the detection of GFP-positive CEM cells by flow-cytometry in HIV-infected cells also infected with the the HIV-dependent expression construct (HDEC) of SEQ ID NO:2 packaged into a lentivirus pseudo-typed with the VSV glycoprotein. Top: CEM cells infected only with HDEC reporter virus. Middle: CEM cells infected only with an HIV where the Nef gene was replaced by the murine CD24. Bottom: CEM cells infected with both HIV and HDEC reporter virus.

### DETAILED DESCRIPTION OF THE INVENTION

The present invention is based, at least in part, on the discovery of novel DNA constructs, referred to herein as “HIV-dependent expression construct”, “HDEC”, or simply “expression construct” nucleic acid molecules, which comprise an expressible sequence whose expression is dependent on the presence of both HIV Tat and Rev proteins. HIV Tat regulates transcription of the expressible sequence mRNA. However, because the reporter is contained, at least in part, within an intron, it is spliced out by the cellular splicing machinery unless Rev is present. Accordingly, these novel expression constructs are capable of detecting HIV infection and/or gene expression with both specificity and sensitivity. They may also be useful in screening assays for compounds capable of inhibiting HIV infection and/or gene expression. They may also be useful for killing HIV-infected cells through the use of cytotoxic expressible sequences.

The HIV-dependent expression constructs of the invention comprise an expressible sequence expressed under the control of (i.e., operably linked to) an HIV-dependent promoter, for example, the HIV 5′ LTR. The constructs further contain at least one splice acceptor-donor site pair and a Rev Responsive Element (RRE). When the HIV-dependent expression constructs are introduced into a cell, any mRNA transcribed from the expressible sequence will be spliced out if Rev is not present. However, when Rev is present (e.g., when the cell is infected with HIV), it will act through the RRE to prevent splicing of the expressible sequence. The expressible sequence can then be detected, either by detecting the mRNA or the encoded protein directly, or by detecting the activity of the encoded protein.

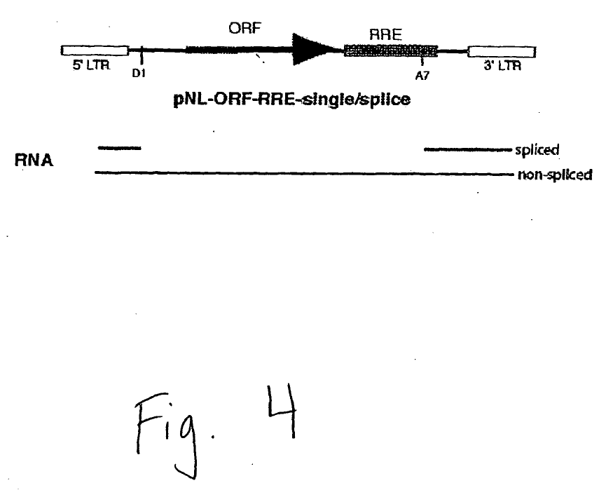

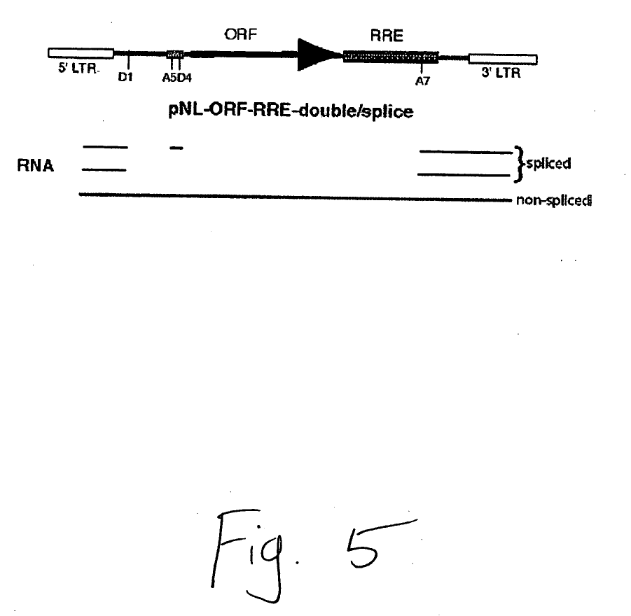

Schematic diagrams of two non-limiting exemplary embodiments of the HIV-dependent expression constructs of the invention are shown in FIGS. 4 and 5. The two ends of the construct, are equivalent to the termini of the linear HIV genome. The central region is composed of an expressible sequence. Expression from this expressible sequence following integration of the construct into the host cell genome is dependent on Tat and Rev expression from an alternative source (e.g., infecting HIV).

As used herein, the term “expressible sequence”, includes any nucleic acid sequence, preferably a DNA sequence, that, when operably linked a to a promoter, is capable of being transcribed to produce complementary RNA. In a preferred embodiment, an expressible sequence is a reporter gene and/or a therapeutic gene, as described herein. In some embodiments, an HIV-dependent expression vector of the invention may comprise an expressible sequence which itself comprises multiple reporter and/or therapeutic genes, which may be linked in frame, or which may be separated by other nucleic acid sequences within the construct.

As used herein, the term “operably linked” is intended to mean that the expressible sequence is linked to the promoter, in a manner which allows for expression of the expressible sequence (e.g., in an in vitro transcription/translation system or in a host cell). Additionally, the term “operably linked” is intended to include the linkage order of the various elements of the HIV-dependent expression constructs, as described herein, such that the HIV-dependent expression constructs perform according to their intended function, as described herein.

As used herein a “reporter” or a “reporter gene” refers to a nucleic acid molecule capable of being transcribed as mRNA when operatively linked to a promoter (e.g., an HIV-derived promoter such as the HIV 5′ LTR), except that the term “reporter gene” as used herein, is not intended to include wild-type HIV sequences. Preferred reporter genes include luciferase (e.g., firefly luciferase or Renilla luciferase), β-galactosidase, chloramphenicol acetyl transferase (CAT), thymidine kinase (TK), and fluorescent proteins (e.g., green fluorescent protein, red fluorescent protein, yellow fluorescent protein, blue fluorescent protein, cyan fluorescent protein, or variants thereof, including enhanced variants).

Any reporter nucleic acid sequence may be used as a reporter gene if is it is detectable by a reporter assay. Reporter assays include any known method for detecting a nucleic acid :sequence or its encoded protein product directly or indirectly. For example, a reporter assay can measure the level of reporter gene expression or activity by measuring the level of reporter mRNA, the level of reporter protein, or the amount of reporter protein activity. The level of reporter mRNA may be measured, for example, using ethidium bromide staining of a standard RNA gel, Northern blotting, primer extension, or nuclease protection assay. The level of reporter protein may be measured, for example, using Coomassie staining of an SDS-PAGE gel, Western blotting, dot blotting, slot blotting, ELISA, or RIA. Reporter protein activity may be measured using an assay specific to the reporter being used. For example, standard assays for luciferase, CAT, β-galactosidase, thymidine kinase (TK) assays (including full body scans; see Yu, Y. et al. (2000) Nature Medicine 6:933-937 and Blasberg, R. (2002) J. Cereb. Blood Flow Metab. 22:1157-1164), and fluorescent proteins are all well-known in the art.

It should also be understood that the terms “reporter gene” and “reporter” are intended to include therapeutic genes, including cytotoxic proteins. As used herein, a “therapeutic gene” or “therapeutic protein” includes any gene or protein (e.g., peptide or polypeptide) that, when expressed in the cell, has an effect on the function of the cell. In a preferred embodiment, a therapeutic protein is a protein that is toxic to cells (i.e., cytotoxic). Preferred cytotoxic proteins include, but are not limited to, ricin, pokeweed toxin, diphtheria toxin A. saporin, gelonin, and Pseudomonas exotoxin A. Therapeutic genes also include nucleic acid sequences that encode anti-sense RNAs (which may be used, for example, to inactivate other mRNAs in a cell) and enzymatic RNAs such as ribozymes. Therapeutic genes further may include ribosome-inactivating proteins (Peumans, W. J. et al. (2001) Faseb J. 15:1493-1506)

As used herein, the term “promoter” generally refers a region of genomic DNA, usually found 5′ to an mRNA transcription start site. Promoters are involved in regulating the timing and level of mRNA transcription and contain, for example, binding sites for cellular proteins such as RNA polymerase and other transcription factors. Further description of promoters can be found, for example, in Goeddel (1990) Methods Enzymol. 185:3-7.

The promoters used in the HIV-dependent expression constructs of the present invention preferably are dependent on the presence of HIV Tat protein. A preferred promoter of used in the constructs of the invention is the HIV 5′ LTR. In one embodiment, the promoter includes the entire HIV 5′ LTR. In another embodiment, the promoter includes a fragment of the HIV 5′ LTR. Such a fragment must include at least the minimal sequences required to initiate mRNA transcription in response to HIV Tat protein. See Wu, Y. and Marsh, J. W. (2003) Microbes and Infection 5:1023-1027; Pereira, L. A. et al. (2000) Nucleic Acids. Res. 28:663-668.

Additionally, if the HIV-dependent expression vectors are intended to be included in a recombinant retrovirus, the 5′ and 3′ LTRs are essential for reverse transcription (formation of DNA), integration (in concert with HIV integrase), as well as transcription of the integrated DNA, and generation of the reporter gene. A region of the genome adjacent to the 5′-LTR (called the psi (φ) site) is necessary for incorporation of the vector into the recombinant retrovirus.

The splicing sites (donor and acceptor) are necessary for removal (and silencing) of the expressible sequence. Rev prevents the splicing, and thus promotes expression from the open reading frame. The single-splice construct is the minimum number of sites in a Rev-dependent vector. The two-splice construct is similar to the sites that result in Nef transcript. The doubly spliced Nef transcript is the predominant message in HIV infection, and thus HIV utilizes the favored splice sites in human cells.

The Rev Responsive Element (RRE) is necessary for Rev binding and activity. The vector needs to be incorporated into a recombinant retrovirus in order to be able to infect and become integrated in the targeted cell. For this to occur there are numerous viral proteins that must be supplied in trans to complete an infectious particle capable of a single infection cycle. A system for construction of an HIV-like particle has previously been described.

Accordingly, in a preferred embodiment, the invention provides isolated nucleic acid molecules comprising: a promoter, wherein the activity of the promoter is dependent on the presence of the human immunodeficiency virus (HIV) Tat protein (e.g., the HIV 5′ LTR); at least one splice donor site (e.g., the human HIV D1 splice donor site) and at least one splice acceptor site (e.g., the human HIV A7 splice donor site); an expressible sequence which is not a wild-type HIV sequence, wherein at least part of the expressible sequence is located in an intron between the splice acceptor site and the splice donor site; and a Rev Responsive Element (RRE) from the human immunodeficiency virus. In another preferred embodiments, the nucleic acid molecules of the invention further comprise the human HIV 3′ LTR. In one embodiment, the splice acceptor site is contained within the RRE.

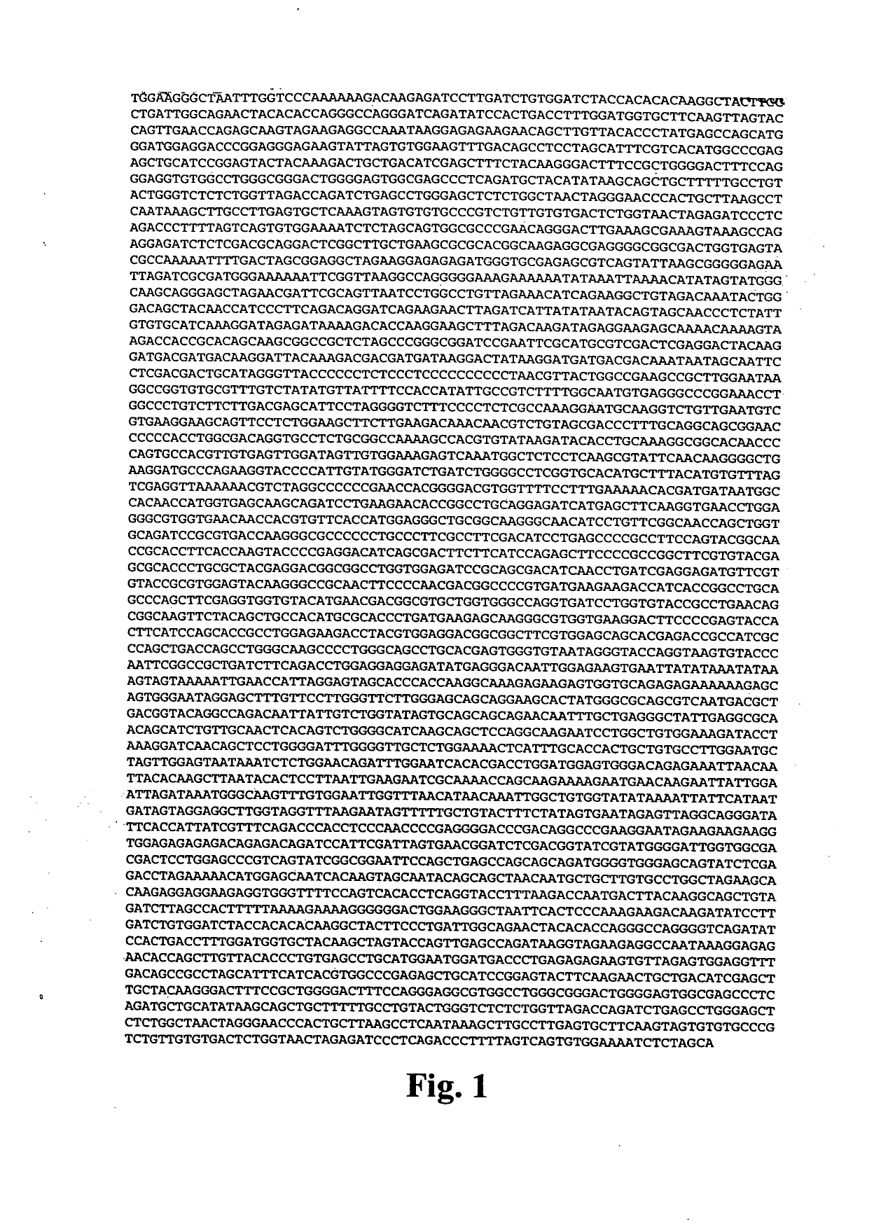

In one embodiment, an isolated nucleic acid molecule of the invention comprises the nucleotide sequence shown in SEQ ID NO:1 (FIG. 1). This nucleic acid molecule comprises a GFP reporter gene flanked by a single splice donor site and a single splice acceptor site, as well as the HIV 5′ and 3′ LTRs. The splice acceptor site is contained within the Rev responsive element. Nucleotides 1-634 of SEQ ID NO:1 comprise the HIV 5′ LTR. Nucleotides 686-823 of SEQ ID NO:1 comprise a genomic RNA packaging signal. Nucleotides 743-744 of SEQ ID NO:1 comprise a splice donor site. Nucleotides 1143-1191 of SEQ ID NO:1 comprise a multiple cloning site. Nucleotides 1299-1-873 of SEQ ID NO:1 comprise an IRES. Nucleotides 1883 2559of SEQ ID NO:1 comprise an open reading frame encoding green fluorescent protein (GFP). Nucleotides 2638-3495 of SEQ ID NO:1 comprise the HIV RRE. Nucleotides 3394-3395 of SEQ ID NO:1 comprise a splice acceptor site. Nucleotides 3784-4418 of SEQ ID NO:1 comprise the HIV 3′ LTR.

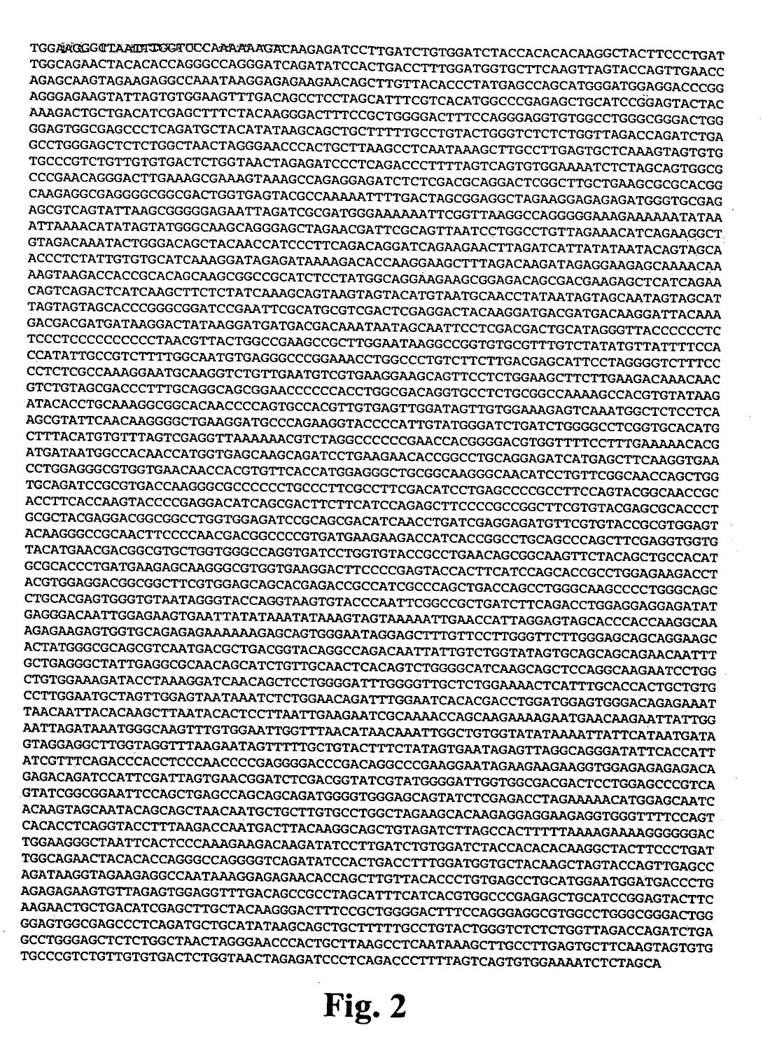

In another embodiment, an isolated nucleic acid molecule of the invention comprises the nucleotide sequence shown in SEQ ID NO:2 (FIG. 2). This nucleic acid molecule comprises a GFP reporter gene, as well as two splice donor sites and two splice acceptor sites and the HIV 5′ and 3′ LTRs. One splice acceptor site is contained within the Rev responsive element. Nucleotides 1-634 of SEQ ID NO:2 comprise the HIV 5′ LTR. Nucleotides 686-823 of SEQ ID NO:2 comprise a genomic RNA packing signal. Nucleotides 743-744 of SEQ ID NO:2 comprise a splice donor site. Nucleotides 1164-1165 of SEQ ED NO:2 comprise a splice acceptor site. Nucleotides 1233-1234 of SEQ ID NO:2 comprise a splice donor site. Nucleotides 1292-1327 of SEQ ID NO:2 comprise Multiple Cloning Site. Nucleotides 1435-2009 of SEQ ID NO:2 comprise an IRES. Nucleotides 2019-2735 of SEQ ID NO:2 comprise an open reading frame encoding green fluorescent protein (GFP). Nucleotides 2774-3631 of SEQ ID NO:2 comprise the HIV RRE. Nucleotides 3530-3531 of SEQ ID NO:2 comprise a splice acceptor site. Nucleotides 3921-4554 of SEQ ID NO:2 comprise the HIV 3′ LTR.

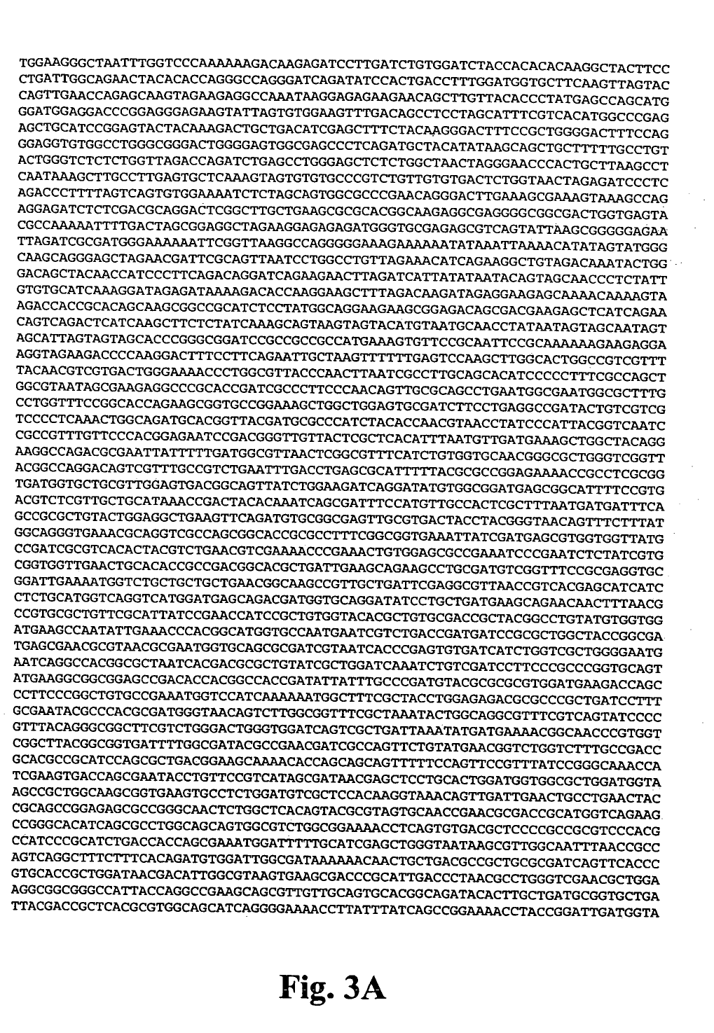

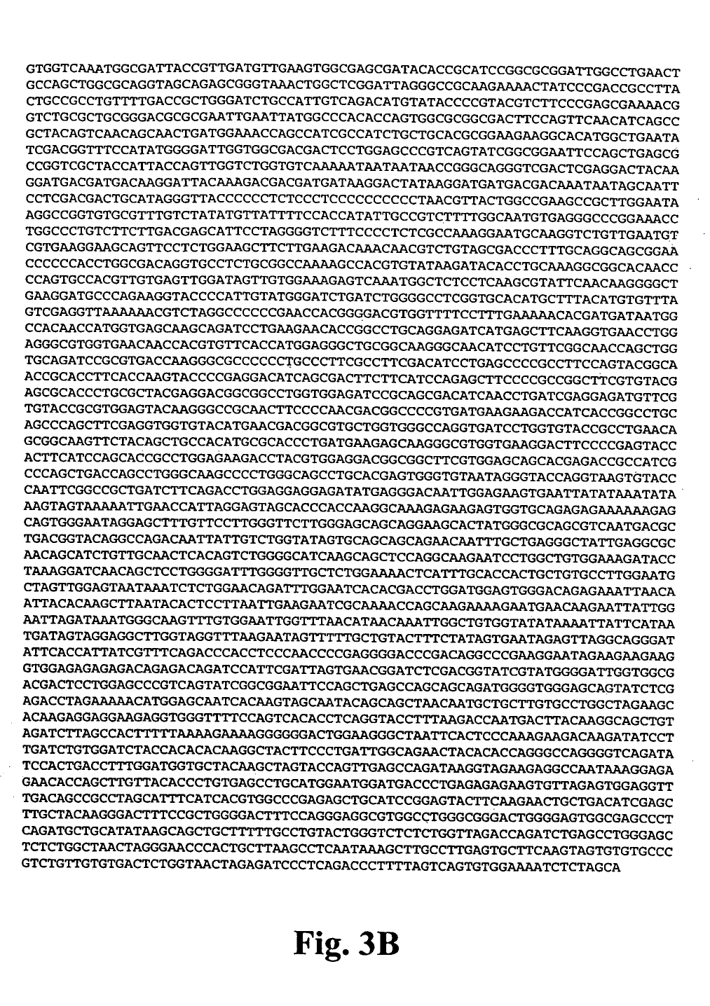

In still another embodiment, an isolated nucleic acid molecule of the invention comprises the nucleotide sequence shown in SEQ ID NO:3 (FIGS. 3A-3B). This nucleic acid molecule comprises a GFP reporter gene and a β-galactosidase reporter gene, as well as two splice donor sites and two splice acceptor sites and the HIV 5′ and 3′ LTRs. One splice acceptor site is contained within the Rev responsive element. Nucleotides 1-634 of SEQ ID NO:3 comprise the HIV 5′ LTR. Nucleotides 686-823 of SEQ ID NO:3 comprise a genomic RNA packing signal. Nucleotides 1-634 of SEQ ID NO:3 comprise the HIV 5′ LTR. Nucleotides 686-823 of SEQ ID NO:3 a genomic RNA packaging signal. Nucleotides 743-744 of SEQ ID NO:3 a splice donor site. Nucleotides 1164-1165 of SEQ ID NO:3 a splice acceptor site. Nucleotides 1233-1234 of SEQ ID NO:3 a splice donor site. Nucleotides 1314-4463 of SEQ ID NO:3 an open reading frame encoding β-galactosidase (lacZ). Nucleotides 4600-5174 of SEQ ID NO:3 an IRES. Nucleotides 5184-5900 of SEQ ID NO:3 an open reading frame encoding green fluorescent protein (GFP). Nucleotides 5939-6796 :of SEQ ID NO:3 the HIV RRE. Nucleotides 6695-6696 of SEQ ID NO:3 a splice acceptor site. Nucleotides 7086-7719 of SEQ ID NO:3 the HIV 3′ LTR.

A plasmid comprising the nucleic acid sequence of SEQ ID NO:1 (nucleotides 1-4418) was deposited with the NIH AIDS Research and Reference Reagent Program, McKesson BioServices Corporation, 621 Lofstrand Lane, Rockville, Md. 20850, on ______, and assigned Accession No. ______. A host cell comprising the nucleic acid sequence of SEQ ID NO:1 (nucleotides 1-4418) was deposited with the NIH AIDS Research and Reference Reagent Program, McKesson BioServices Corporation, 621 Lofstrand Lane, Rockville, Md. 20850, on ______, and assigned Accession No. ______.

A plasmid comprising the nucleic acid sequence of SEQ ID NO:1 (nucleotides 1-4418) was deposited with the American Type Culture Collection (ATCC), 10801 University Boulevard, Manassas, Va. 20110-2209, on ______, and assigned Accession No. ______. A host cell comprising the nucleic acid sequence of SEQ ID NO:1 (nucleotides 1-4418) was deposited with the American Type Culture Collection (ATCC), 10801 University Boulevard, Manassas, Va. 20110-2209, on ______, and assigned Accession No. ______.

A plasmid comprising the nucleic acid sequence of SEQ ID NO:2 (nucleotides 1-4554) was deposited with the NIH AIDS Research and Reference Reagent Program, McKesson BioServices Corporation, 621 Lofstrand Lane, Rockville, Md. 20850, on ______, and assigned Accession No. ______. A host cell comprising the nucleic acid sequence of SEQ ID NO:2 (nucleotides 1-4554) was deposited with the NIH AIDS Research and Reference Reagent Program, McKesson BioServices Corporation, 621 Lofstrand Lane, Rockville, Md. 20850, on ______, and assigned Accession No. ______.

A plasmid comprising the nucleic acid sequence of SEQ ID NO:2 (nucleotides 1-4554) was deposited with the American Type Culture Collection (ATCC), 10801 University Boulevard, Manassas, Va. 20110-2209, on ______, and assigned Accession No. ______. A host cell comprising the nucleic acid sequence of SEQ ID NO:2 (nucleotides 1-4554) was deposited with the American Type Culture Collection (ATCC), 10801 University Boulevard, Manassas, Va. 20110-2209, on ______, and assigned Accession No. ______.

A plasmid comprising the nucleic acid sequence of SEQ ID NO:3 (nucleotides 1-7719) was deposited with the NIH AIDS Research and Reference Reagent Program; McKesson BioServices Corporation, 621 Lofstrand Lane, Rockville, Md. 20850, on ______, and assigned Accession No. ______. A host cell comprising the nucleic acid sequence of SEQ ID NO:3 (nucleotides 1-7719) was deposited with the NIH AIDS Research and Reference Reagent Program, McKesson BioServices Corporation, 621 Lofstrand Lane, Rockville, Md. 20850, on ______, and assigned Accession No. ______.

A plasmid comprising the nucleic acid sequence of SEQ ID NO:3 (nucleotides 1-7719) was deposited with the American, Type Culture Collection (ATCC), 10801 University Boulevard, Manassas, Va. 20110-2209, on ______, and assigned Accession No. ______. A host cell comprising the nucleic acid sequence of SEQ ID NO:3 (nucleotides 1-7719) was deposited with the American Type Culture Collection (ATCC), 10801 University Boulevard, Manassas, Va. 20110-2209, on ______, and assigned Accession No. ______.

The above-referenced deposits will be maintained under the terms of the Budapest Treaty on the International Recognition of the Deposit of Microorganisms for the Purposes of Patent Procedure. These deposits were made merely as a convenience for those of skill in the art and are not admissios that a deposit is required under 35 U.S.C. §112.

I. Isolated Nucleic Acid Molecules

One aspect of the invention pertains to isolated nucleic acid molecules that comprise the HIV-dependent expression constructs. As used herein, the term ‘nucleic acid molecule’ is intended generally to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

In general, optimal practice of the present invention can be achieved by use of recognized manipulations. For example, techniques for isolating mRNA, methods for making and screening cDNA libraries, purifying and analyzing nucleic acids, methods for making recombinant vector DNA, cleaving DNA with restriction enzymes, ligating DNA, introducing DNA into host cells by stable or transient means, culturing the host cells, methods for isolating and purifying polypeptides and making antibodies are generally known in the field. See generally Sambrook et al., Molecular Cloning (2d ed. 1989): and Ausubel et al., Current Protocols in Molecular Biology, (1989) John Wiley & Sons, New York.

The term ‘isolated nucleic acid molecule’ includes nucleic acid molecules which are separated from other nucleic acid molecules which are present in the natural source of the nucleic acid. For example, with regards to genomic DNA, the term ‘isolated’ includes: nucleic acid molecules which are separated from the viral DNA or chromosome with which the genomic DNA is naturally associated. Preferably, an ‘isolated’ nucleic acid is free of sequences which naturally flank the nucleic acid (i.e., sequences-located at the 5′ and 3′ ends of the nucleic acid) in the genomic DNA of the organism from which the nucleic acid is derived. For example, in various embodiments, the isolated HIV-dependent expression construct nucleic acid molecule can contain less than about 5 kb, 4 kb, 3 kb, 2 kb, 1 kb, 0.5 kb or 0. 1 kb of nucleotide sequences which naturally flank the nucleic acid molecule in genomic DNA of the HIV virus from which the nucleic acid is derived. Moreover, an ‘isolated’ nucleic acid molecule can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, 2, or 3, or a portion thereof, can be constructed using standard molecular biology techniques and the sequence information provided herein.

Moreover, a nucleic acid molecule encompassing all or a portion of SEQ ID NO:1, 2, or 3 can be isolated by the polymerase chain reaction (PCR) using synthetic oligonucleotide primers designed based upon the sequence of SEQ ID NO:1, 2, or 3.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to HIV-dependent expression construct nucleotide sequences can be prepared by standard synthetic techniques, e.g., using an automated DNA synthesizer.

In still another embodiment,: an isolated nucleic acid molecule of the invention comprises a nucleic acid molecule which is a complement of the nucleotide sequence shown in SEQ ID NO:1, 2, or 3. A nucleic acid molecule which is complementary to the nucleotide sequence shown in SEQ ID NO:1, 2, or 3 is one which is sufficiently complementary to the nucleotide sequence shown in SEQ ID NO:1, 2, or 3 such that it can hybridize to the nucleotide sequence shown in SEQ ID NO:1, 2, or 3, thereby forming a stable duplex. The term ‘complementary’ or like term refers to the hybridization or base pairing between nucleotides or nucleic acids, such as, for instance, between the two strands of a double stranded DNA molecule or between an oligonucleotide primer and a primer binding site on a single stranded nucleic, acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 95% of the nucleotides of the other strand, usually at least about 98%, and more preferably from about 99 to about 100%. Complementary polynucleotide sequences can be identified by a variety of approaches including use of well-known computer algorithms and software.

In still another embodiment, an isolated nucleic acid molecule of the present invention comprises a nucleotide sequence which is at least about 50%, 55%, 60%, 65%, 70%, 75%, 80%,85%,90%,91%,92%,93%,94%,95%,96%,97%,98%,99%,99.1%, 99.2%, 99.3%, 99.4%, 99.5%, 99.6%, 99.7%, 99.8%, 99.9% or more identical to the nucleotide sequence shown in SEQ ID NO:1, 2, or 3 (e.g., to the entire length of the nucleotide sequence), or a portion or complement of any of these nucleotide sequences. In one embodiment, a nucleic acid molecule of the present invention comprises a nucleotide sequence which comprises part or all of SEQ ID NO:1 or 2, or a complement thereof, and which is at least (or no greater than) 25, 30, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1250, 1300, 1350, 1400, 1450, 1500, 1550, 1600, 1650, 1700, 1750, 1800, 1850, 1900, 1950, 1994, 2000, 2050, 2073, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3441, 3450, 3500, 3550, 3600, 3650, 3700, 3750, 3800, 3841, 3850, 3900, 3950; 4000, 4050, 4100, 4150, 4200, 4250, 4300, 4350, 4400, 4450, 4500, 4550, 4600, 4650, 4700, 4750, 4800, 4850, 4900, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200; 7250, 7300, 7350, 7400, 7450, 7500, 7550, 7600, 7650, 7700 or more nucleotides (e.g contiguous nucleotides) in length.

To determine the percent identity of two nucleic acid or amino acid sequences, the sequences are aligned for optimal comparison purposes (e.g., gaps can be introduced in one, or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to a nucleotide sequence having 100 nucleotides, at least 30, preferably at least 40, more preferably at least 50, even more preferably at least 60, and even more preferably at least 70, 80, or 90 nucleotides are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at online through the Genetics Computer Group), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at online through the Genetics Computer Group), using a NWSgapdna.CMP matrix and a gap weight of 40, 50, 60, 70, or 80 and a length weight of 1, 2, 3, 4, 5, or 6. A preferred, non-limiting example of parameters to be used in conjunction with the GAP program include a Blossum 62 scoring matrix with a gap penalty of 12, a gap extend penalty of 4, and a frameshift gap penalty of 5.

In another embodiment, the percent identity between two amino acid or nucleotide sequences is determined using the algorithm of Meyers and Miller (Comput. Appl. Biosci. 4:11-17 (1988)) which has been incorporated into the ALIGN program (version 2.0 or version 2.0 U), using a PAM120 weight residue table, a gap length penalty of 12 and a gap penalty of 4.

The nucleic acid molecule of the invention can comprise only a portion of the nucleic acid sequence of SEQ ID NO:1, 2, or 3, for example, a fragment which can be used as a probe or primer or a fragment encoding a portion of an HIV-dependent expression construct. The probe/primer (e.g., oligonucleotide) typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of SEQ ID NO: 1, 2, or 3, or a complement thereof.

Exemplary probes or primers are at least (or no greater than) 12 or 15, 20 or 25, 30, 35, 40, 45, 50, 55, 60, 65, 70, 75 or more nucleotides in length and/or comprise consecutive nucleotides of an isolated nucleic acid molecule described herein. Also included within the scope of the present invention are probes or primers comprising contiguous or consecutive nucleotides of an isolated nucleic acid molecule described herein, but for the difference of 1, 2, 3, 4, 5, 6, 7, 8, 9 or 10 bases within the probe or primer sequence. Probes based on the HIV-dependent expression construct nucleotide sequences can be used to detect (e.g., specifically detect) genomic sequences. In preferred embodiments, the probe further comprises a label group attached thereto, e.g., the label group can be a radioisotope, a fluorescent compound, an enzyme, or an enzyme co-factor. In another embodiment a set of primers is provided, e.g., primers suitable for use in a PCR, which can be used to amplify a selected region of an HIV-dependent expression construct sequence, e.g., a domain, region, site or other sequence described herein. The primers should be at least 5, 10, or 50 base pairs in length and less than 100, or less than 200, base pairs in length. The primers should be identical, or differ by no greater than 1, 2, 3, 4, 5, 6, 7, 8, 9 or 10 bases when compared to a sequence disclosed herein or to the sequence of a naturally occurring variant. Such probes can be used as a part of a diagnostic test kit for identifying cells or tissue which contain the expression construct, or which:express the expressible sequence.

In another embodiment, nucleic acid molecules of the invention can comprise variants of the sequence elements disclosed herein. Nucleic acid variants (e.g., variants of the 5′ or 3′ LTRs, the RRE, and/or the splice acceptor sites) can be naturally occurring, such as allelic variants (same locus), homologues (different locus), and orthologues (different organism, e.g., mouse) or can be non-naturally occurring. Non-naturally occurring variants can be made by mutagenesis techniques, including those applied to polynucleotides, cells, or organisms. The variants can contain nucleotide substitutions, deletions, inversions and insertions. Allelic variants result, for example, from DNA sequence polymorphisms within a population (e.g., the HIV population).

Nucleic acid molecules corresponding to natural allelic variants and homologues of the individual elements of the HIV-dependent expression constructs of the invention can be isolated based on their homology to the HIV-dependent expression construct nucleic acids disclosed herein using the nucleic acid sequences disclosed herein, or a portions thereof, as hybridization probes according to standard hybridization techniques under stringent hybridization conditions.

As used herein, the term ‘hybridizes under stringent conditions’ is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4, and 6. Additional stringent conditions can be found in Molecular Cloning: A Laboratory Manual, Sambrook et al., Cold Spring Harbor Press, Cold Spring Harbor, N.Y. (1989), chapters 7, 9, and 11. A preferred, non-limiting example of stringent hybridization conditions includes hybridization in 4 X sodium chloride/sodium citrate (SSC), at about 65-70° C. (or alternatively hybridization in 4X SSC plus 50% formamide at about 42-50° C.) followed by one or more washes in IX SSC, at about 65-70° C. A preferred, non-limiting example of highly stringent hybridization conditions includes hybridization in 1X SSC, at about 65-70° C. (or alternatively hybridization in IX SSC plus 50% formamide at about 42-50° C.) followed by one or more washes in 0.3X SSC, at about 65-70° C. A preferred, non-limiting example of reduced stringency hybridization conditions includes hybridization in 4X SSC at about 50-60° C. (or alternatively hybridization in 6X SSC plus 50% formamide at about 40-45° C.) followed by one or more washes in 2X SSC, at about 50-60° C. Ranges intermediate to the above-recited values, e.g., at 65-70° C. or at 42-50° also intended to be encompassed by the present invention. SSPE (1X SSPE is 0.15M NaCl, 10 mM NaH₂PO₄, and 1.25 mM EDTA, pH 7.4) can be substituted for SSC (1X SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1X SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., ED TA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (see e.g., Church and Gilbert (1984) Proc. Natl. Acad. Sci. USA 81:1991 1995), or alternatively 0.2X SSC, 1% SDS.

In addition to naturally-occurring allelic variants of the elements of the HIV-dependent expression construct sequences that may exist in the population, the skilled artisan will further appreciate that changes can be introduced by mutation into the nucleotide sequences of SEQ ID NO:1, 2, or 3, without altering the functional ability of the HIV-dependent expression construct sequences. In one embodiment, the isolated nucleic acid molecule comprises a nucleotide sequence which is at least about 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more identical to SEQ ID NO:1, 2, or 3, e.g., to the entire length of SEQ ID NO:1, 2, or 3.

II. Recombinant Expression Vectors and Host Cells

Another aspect of the invention pertains to vectors, for example recombinant expression vectors, containing an HIV-dependent expression construct nucleic acid molecule. As used herein, the term ‘vector’ refers to a nucleic acid molecule capable of transporting another nucleic acid to which it has been linked. One type of vector is a ‘plasmid’, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as ‘expression vectors’. In general, expression. vectors of utility in recombinant DNA techniques are often in the form of plasmids. In the present specification, ‘plasmid’ and ‘vector’ can be used interchangeably as the plasmid is the most commonly used form of vector. However, the invention is intended to include such other forms of expression vectors, such as viral vectors (e.g., replication defective retroviruses, adenoviruses, adeno-associated viruses, and lentiviruses), which serve equivalent fuictions.

In a preferred embodiment, the HIV-dependent expression constructs are contained within a retroviral vector, which can be used to infect mammalian cells (e.g., human cells).

In a more preferred embodiment, the retroviral vector is replication incompetent. This is particularly important because it would be highly undesirably to produce a replication competent retrovirus that contains HIV sequences, which could potentially infect humans and cause disease.

A particularly preferred retroviral vector for the expression of the HIV-dependent expression constructs are the lentiviral vectors described in Naldini, L. et al. ((1996) Science 272:263-267, incorporated herein by reference). Lentiviral vectors are particularly useful for detecting HIV infection in non-dividing (as well as dividing) cells. Other preferred vectors are described in U.S. Pat. Nos. 6,428,953, 6,165,782, 6,013,516, and 5,994,136, all of which are incorporated herein by reference.

Another aspect of the invention pertains to host cells into which an HIV-dependent expression construct nucleic acid molecule of the invention is introduced, e.g., an HIV-dependent expression construct nucleic acid molecule within a vector (e.g., a recombinant retroviral vector) or an HIV-dependent expression construct nucleic acid molecule containing sequences which allow it to homologously recombine into a specific site of the host cell's genome. The terms ‘host cell’ and ‘recombinant host cell’ are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a vector containing an HIV-dependent expression construct can be propagated and/or expressed in bacterial cells such as E. coli, insect cells, yeast or mammalian cells (such as Chinese hamster ovary cells (CHO), COS cells (e.g., COS7 cells), C6 glioma cells, HEK 293T cells, or neurons). Other suitable host cells are known to those skilled in the art. In a preferred embodiment, a host cell is a human T cell (e.g., a CEM T cell).

Vector DNA can be introduced into prokaryotic or eukaryotic cells via conventional transformation or transfection techniques. As used herein, the terms ‘transformation’ and ‘transfection’ are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an HIV-dependent expression construct or can be introduced on a separate vector. Cells stably transfected with the introduced nucleic acid can be identified by drug selection (e.g., cells that have incorporated the selectable marker gene will survive, while the other cells die).

In a most preferred embodiment, host cells containing the HIV-dependent expression constructs of the invention are produced by infecting cells with a recombinant retrovirus containing the constructs. Preferred method for the production of host cells can be found, for example, in Naldini et al. ((1996) supra and in U.S. Pat. Nos. 6,428,953, 6,165,782, 6,013,516, and 5,994,136, all of which are incorporated herein by reference.

The host cells of the invention can also be used to produce non-human transgenic animals. For example, in one embodiment, a host cell of the invention is a fertilized oocyte or an embryonic stem cell into which HIV-dependent expression construct sequences have been introduced. Such host cells can then be used to create non-human transgenic animals in which exogenous HIV-dependent expression construct sequences have been introduced into their genome. Such animals are useful for studying HIV infection and/or gene expression and for identifying and/or evaluating modulators of HIV infection and/or gene expression. As used herein, a ‘transgenic animal’ is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal.

A transgenic animal of the invention can be created by introducing an HIV-dependent expression construct-encoding nucleic acid into the male pronuclei of a fertilized oocyte, e.g., by microinjection or retroviral infection, and allowing the oocyte to develop in a pseudopregnant female foster animal. The HIV-dependent expression construct sequence of SEQ ID NO:1, 2, or 3 can be introduced as a transgene into the genome of a non-human animal. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of an HIV-dependent expression construct transgene in its genome and/or expression of the expressible sequence of the HIV-dependent expression construct transgene in tissues or cells of the animals. A transgenic founder animal can then be used to breed additional animals carrying the transgene. Moreover, transgenic animals carrying a transgene containing an HIV-dependent expression construct can further be bred to other transgenic animals carrying other transgenes.

Transgenic animals of the invention can also be used to produce stable cell lines containing the HIV-dependent expression construct. Such cell lines are useful because they can be made so that they do not overexpress the transgene (as may happen in transient transfection), and therefore more closely reflect the natural cellular environment of the transgene. Such cell lines may be produced by isolating cells (e.g., T cells cells) from a transgenic animal (e.g., a mouse) and culturing them using standard methods. In some embodiments primary (i.e., non-immortalized) cells are preferred, or the cells may be may be immortalized (e.g., by the addition of a gene such as SV40 large T antigen) in order to propagate them indefinitely in culture.

III. Methods of Detecting HIV

In still another embodiment the invention provides a method of determining whether HIV is present in a sample comprising: contacting a host cell containing a nucleic acid molecule of the invention with the sample; culturing the cell for an amount of time sufficient to allow HIV infection and gene expression; and determining whether the expressible sequence is expressed by the cell, wherein expression of the expressible sequence is indicative of the presence of HIV in the sample. In a preferred embodiment, the biological sample is isolated from a subject (e.g., a human subject). In a further preferred embodiment, the biological sample is selected from the group consisting of a biological fluid sample (e.g., blood, serum, plasma, saliva, urine, stool, semen, vaginal fluid, spinal fluid, lymph, amniotic fluid, tears, nasal secretions, sweat, breast milk, mucus, or interstitial fluid), a tissue sample (e.g., a lymph node sample, a skin sample, or a chorionic villus sample), and a cell sample (e.g., a blood cell sample such as a T cell sample). In a further embodiment, the sample may be purified.

In another embodiment, the invention provides a method of determining whether a cell (e.g., a T cell) is infected with HIV comprising: contacting the cell with the retrovirus containing a nucleic acid molecule of the invention; culturing the cell for an amount of time sufficient to allow HIV gene expression; and determining whether the expressible sequence is expressed by the cell, wherein expression of the expressible sequence is indicative of HIV infection of the cell.

In yet another embodiment, the invention provides a method of determining whether a subject (e.g., a human subject) is infected with HIV comprising contacting the cells of the subject with a retrovirus containing a nucleic acid molecule of the invention, and determining whether the expressible sequence is expressed by the cells, wherein expression of the expressible sequence is indicative of HIV infection.

IV. Screening Assays

The invention provides a method (also referred to herein as a “screening assay”) for identifying modulators, i.e., candidate or test compounds or agents (e.g., nucleic acids, peptides, peptidomimetics, small molecules, or other drugs) which can inhibit HIV infection and/or gene expression.

The screening assays of the invention rely of the ability of the HIV-dependent expression constructs described herein to detect HIV infection. Because the expressible sequence is only expressed when both Tat and Rev are present, host cells containing the expression constructs of the invention can be infected with HIV and tested to identify compounds which can inhibit HIV infection and/or gene expression.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1 997) Anticancer Drug Des. 12:45).

Examples of methods for the synthesis of molecular libraries can be found in the art, for example, in: DeWitt et al. (1993) Proc. Natl. Acad. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra).

In one embodiment, the screening assay is a cell based assay comprising contacting a host cell containing an HIV-dependent expression of the invention with a test compound; contacting the cell with HIV; culturing the cell for an amount of time sufficient to allow HIV infection and gene expression; and determining whether the expressible sequence is expressed by the cell. The HIV-dependent expression construct is preferably stably integrated into the genome of the cell. The test compound may be added prior to, at the same time as, or subsequent to HIV infection of the cell.

In another embodiment, the screening assay of the invention is a cell-based assay comprising contacting a cell with HIV; contacting the cell with a retrovirus containing an HIV-dependent expression construct of the invention; contacting the cell with a test compound; culturing the cell for an amount of time sufficient to allow HIV infection and gene expression; and determining whether the expressible sequence is expressed by the cell. The steps of HIV infection, HIV-dependent expression construct (retrovirus) infection, and test compound addition may be performed at the same time, or in any order.

In still another the screening assay of the invention is a cell-based assay comprising contacting a cell infected with HIV with a retrovirus containing an HIV-dependent expression construct of the invention; contacting the cell with a test compound; culturing the cell for an amount of time sufficient to allow HIV infection and gene expression; and determining whether the expressible sequence is expressed by the cell. The steps of HIV-dependent expression construct (retrovirus) infection and test compound addition may be performed at the same time, or in any order. It should be noted that this embodiment is particularly useful if the host cells used in the screening assay are already infected with HIV.

Determining the ability of the test compound to modulate HIV infection and/or gene expression is accomplished by monitoring expressible sequence expression (e.g., reporter mRNA or polypeptide expression level) or activity, for example. As described elsewhere herein, in the absence of HIV Rev protein, any mRNA expressed from the expressible sequence is splice out as part of an intron, and is not detectable.

The expressible sequence can be a nucleic acid sequence, the expression of which can be measured by, for example, Northern blotting, RT-PCR, primer extension, or nuclease protection assays. The expressible sequence may also be a nucleic acid sequence that encodes a polypeptide, the expression of which can be measured by, for example, Western blotting, ELISA, or RIA assays. Expressible sequence expression can also be monitored by measuring the activity of the polypeptide encoded by the expressible sequence using, for example, a luciferase assay, a β-galactosidase assay, a chloramphenicol acetyl transferase (CAT) assay, a thymidine kinase assay, or a fluorescent protein assay. The methods for performing such assays are well-known in the art.

The level of expression or activity of a expressible sequence under the control of the HIV-dependent expression construct in the presence of the candidate compound is compared to the level of expression or activity of the expressible sequence in the absence of the candidate compound. The candidate compound can then be identified as a modulator of HIV infection and/or gene expression based on this comparison. For example, when expression of expressible sequence mRNA or protein, or protein activity is greater (statistically significantly greater) in the presence of the candidate compound than in its absence, the candidate compound is identified as a stimulator of HIV infection and/or gene expression (undesirably). Preferably, when expression or activity of expressible sequence mRNA or protein is less (statistically significantly less) in the presence of the candidate compound than in its absence, the candidate compound is identified as an inhibitor of HIV infection and/or gene expression.

This invention further pertains to novel agents identified by the above-described screening assays. Accordingly, it is within the scope of this invention to further use an agent identified as described herein in an appropriate animal model (e.g., an HIV infection animal model such as a non-human primate infected with HIV or SIV (simian immunodeficiency virus)). For example, an agent identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

V. Methods of Treatment

The HIV-dependent expression constructs may be used to treat a subject (e.g., a human subject) infected with HIV by using an expressible sequence that encodes a therapeutic protein. As used herein, a “therapeutic protein” is any protein (e.g., peptide or polypeptide) that, when expressed in the cell, has an effect on the function of the cell. In a preferred embodiment, a therapeutic protein is a protein that is toxic to cells (i.e., cytotoxic). Preferred cytotoxic proteins include, but are not limited to, ricin, pokeweed toxin, diphtheria toxin A, saporin, gelonin, and Pseudomonas exotoxin A. Because the expressible sequence in the HIV-dependent expression constructs of the invention is only expressed in the presence of HIV proteins, a cytotoxic protein can be used to selectively kill HIV infected cells. Accordingly, the invention provides method of killing HIV infected cells, as well as methods of treating HIV infected subjects.

As used herein, “treatment” of a subject includes the application or administration of a therapeutic agent (e.g., an HIV-dependent expression construct) to a subject, or application or administration of a therapeutic agent to a cell or tissue from a subject, who has a diseases or disorder (e.g., HIV infection or AIDS), has a symptom of a disease or disorder, or is at risk of (or susceptible to) a disease or disorder, with the purpose of curing, healing, alleviating, relieving, altering, remedying, ameliorating, improving, or affecting the disease or disorder, the symptom of the disease or disorder, or the risk of (or susceptibility to) the disease or disorder.

A cytotoxic protein may be expressed in an HIV infected cell by infecting the cell with a retrovirus containing an HIV-dependent expression construct of the invention in which the expressible sequence is a cytotoxic protein. The cell may be any cell that is infected with HIV, for example a T cell. The cell may be, for example, a cultured cell line, or a cell removed from a subject (e.g., a human subject) by conventional methods.

An HIV-dependent expression construct containing a cytotoxic expressible sequence may also be used to treat a subject (e.g., a human subject) infected with HIV or at risk of being infected with HIV. A retrovirus containing the HIV-dependent expression construct can be administered directly to the subject so that it can infect the cells (e.g., the T cells) of the subject. Once delivered to the cells via the retrovirus, the HIV-dependent expression vector will only express the cytotoxic protein if the cells are or become infected with HIV, thus killing the cells and preventing the virus from replicating and spreading. It should be understood that in any method involving administration of a retrovirus to human subjects, particularly a retrovirus containing HIV-derived sequences, the retrovirus should be replication-incompetent, so that it cannot reproduce after infecting a cell.

In some embodiments, treatment of a subject with an HIV-dependent expression construct of the invention may be administered in-conjunction with other therapies for HIV infection and/or AIDS (e.g., approved or experimental therapies). For example, the HIV-dependent expression vectors of the invention may be administered in conjunction with known AIDS drugs, which include, but are not limited to, protease inhibitors, reverse transcriptase inhibitors, and nucleoside analogs. Examples of such drugs include, but ate not limited to, Agenerase (amprenavir), Combivir (combination of Retrovir (300 mg) and Epivir (150 mg)—together in the same tablet), Crixivan (indinavir), Epivir (3tc/lamivudine), Emtriva (emtricitabine (FTC)), Fortovase (saquinavir), Fuzeon (enfuvirtide), Hivid (ddc/zalcitabine), Hydrea (hydroxyurea), Invirase (saquinavir), Kaletra (lopinavir), Norvir (ritonavir), Rescriptor (delavirdine), Retrovir, AZT (zidovudine), Reyataz (atazanavir; BMS-232632), Sustiva (efavirenz), Trizivir (3 non nucleosides in one tablet; abacavir+zidovudine+lamivudine), Videx (ddl/didanosine), Videx EC; (ddl/didanosine), Viracept (nelfinavir), Viramune (nevirapine), Viread (tenofovir-disoproxil fulmarate), Zerit (d4t/stavudine), and Ziagen (abacavir).

This invention is further illustrated by the following examples which should not be construed as limiting. The contents of all references, patents and published patent applications cited throughout this application, as well as the sequence listing and the figures, are incorporated herein by reference.

### EXAMPLES

### Example 1

### Use of an HIV-Dependent Expression Vector to Detect Cells Infected with HIV

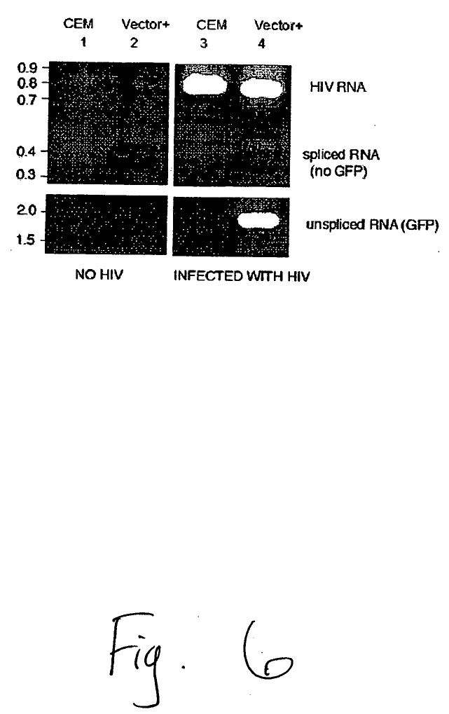

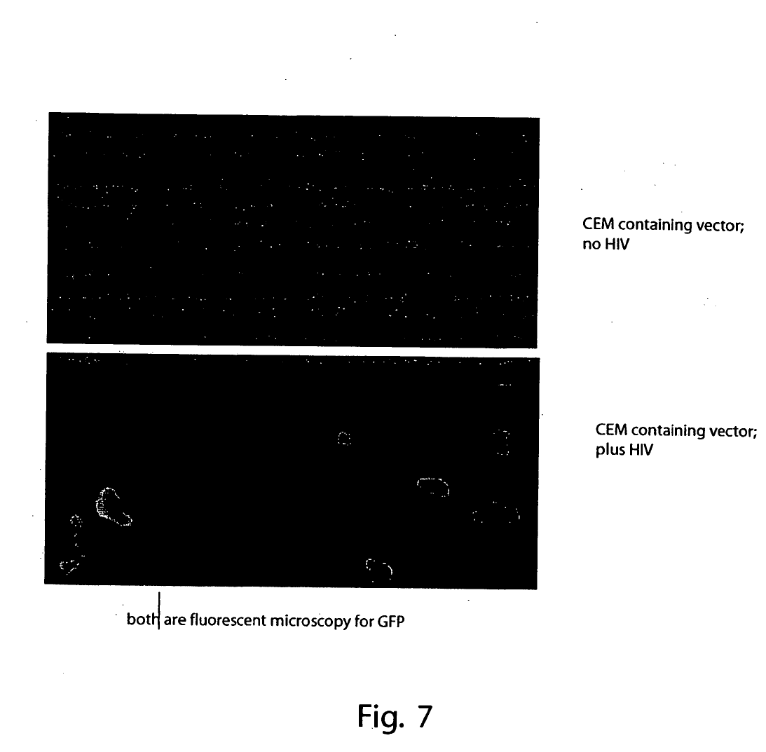

The human T cell line CEM was infected with the HIV-dependent expression construct of SEQ ID NO:2 using the system described by Naldini et al. ((1996) Science 272:263-267, incorporated herein by reference), in which the retroviral vector was replaced with our double-splice vector of SEQ ID NO:2. A cloned cell that possessed a stable integrated form of the HIV-dependent expression construct was examined. The cell expressed RNA from the integrated construct in the absence of Tat (see FIG. 6; spliced RNA; see lane 2) but does not express the GFP-encoding unspliced message. The CEM cell (not containing vector) does not express either RNA (lane 1). Following HIV infection the vector-positive line now expresses high levels of the GFP-encoding RNA (unspliced RNA) in lane 4. Fluorescence microscopy also shows strong GFP fluorescence in HDEC-infected cells when infected with HIV (FIG. 7).

The low level expression of spliced RNA in non-infected cells (no Tat protein) demonstrates the leakiness of the Tat-dependent reporter. The lack of unspliced RNA in the absence of HIV (no Rev protein) demonstrates the selectivity of this system.

### Example 2

### Use of HIV-Dependent Expression Construct Incorporated into a Lentivirus to Detect Actively Infected Cells

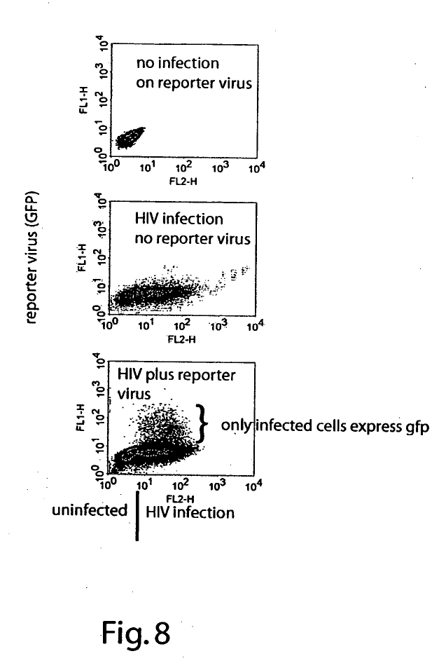

The the HIV-dependent expression construct of SEQ ID NO:2 (also referred to herein as pNL-ORF-RRE-double/splice construct) was packaged into a lentivirus which was pseudo-typed with the VSV glycoprotein, and where the expressible sequence was green fluorescent protein (GFP). Transduction of CEM cells with reporter virus but without HIV infection resulted in no reporter generation (FIG. 8, top, FL1-H).

Human CEM T cells were infected with an HIV where the Nef gene was replaced by the murine CD24. Staining of cells for surface murine CD24 (FL2-H) defined HIV infected cells. (FIG. 8, middle).

Following HIV infection, cells were infected with reporter virus, and examined by flow cytometry. GFP-positive cells (reporter from construct; FL1-H) were found specifically in HIV infected (FL2-H positive) cells only (FIG. 8, bottom).

The invention has been described in detail with reference to preferred embodiments thereof. However, it will be appreciated that those skilled in the art, upon consideration of this disclosure, may make modification and improvements within the spirit and scope of the invention as set forth in the following claims.

1 An isolated nucleic acid molecule comprising:
a) a promoter, wherein the activity of the promoter is dependent on the presence of the human immunodeficiency virus (HIV) Tat protein;
b) at least one splice donor site and at least one splice acceptor site;
c) an expressible sequence which is not a wild-type HIV sequence, wherein at least part of the expressible sequence is located in an intron between the splice acceptor site and the splice donor site; and
d) a Rev Responsive Element (RRE) from the human immunodeficiency virus,
wherein elements (a)-(d) are operably linked; or a complement thereof.

2 The nucleic acid molecule of claim 1, wherein the promoter comprises a human HIV 5′ long terminal repeat (LTR) or a portion thereof; or a complement thereof.

3 The nucleic acid molecule of claim 1, further comprising a human HIV 3′ LTR; or a complement thereof.

4 The nucleic acid molecule of claim 1, wherein the splice donor site is the HIV D1 splice donor site; or a complement thereof.

5 The nucleic acid molecule of claim 1, wherein the splice acceptor site is the HIV A7 splice acceptor site; or a complement thereof.

6 The nucleic acid molecule of claim 1, wherein the splice acceptor site is contained within the RRE; or a complement thereof.

7 The nucleic acid molecule of claim 1, further comprising at least a second splice donor site and at least a second splice acceptor site; or a complement thereof.

8 The nucleic acid molecule of claims 7, wherein the second splice donor site is the HIV D4 splice donor site; or a complement thereof.

9 The nucleic acid molecule of claim 7, wherein the second splice acceptor site is the HIV A5 splice acceptor site; or a complement thereof.

10 The nucleic acid molecule of claim 1, wherein the nucleic acid molecule comprises the nucleic acid molecule depicted in FIG. 4; or a complement thereof.

11 The nucleic acid molecule of claim 1, wherein the nucleic acid molecule comprises the nucleic acid molecule depicted in FIG. 5; or a complement thereof.

12 The nucleic acid molecule of claim 1, further comprising a psi (φ) site; or a complement thereof.

13 The nucleic acid molecule of claim 1, wherein the expressible sequence is a reporter gene; or a complement thereof.

14 The nucleic acid molecule of claim 13, wherein the reporter gene encodes a protein selected from the group consisting of: a fluorescent protein, luciferase, β-galactosidase, chloramphenicol acetyl transferase (CAT), thymidine kinase (TK); or a complement thereof.

15 The nucleic acid molecule of claim 14, wherein the fluorescent protein is selected from the group consisting of green fluorescent protein (GFP), enhanced green fluorescent protein (EGFP), red fluorescent protein (RFP), yellow fluorescent protein (YFP), enhanced yellow fluorescent protein (EYFP), blue fluorescent protein (BFP), and cyan fluorescent protein (CFP); or a complement thereof.

16 The nucleic acid molecule of claim 15, wherein the luciferase is selected from the group consisting of firefly luciferase and Renilla luciferase; or a complement thereof.

17 The nucleic acid molecule of claim 1, wherein the expressible sequence comprises a therapeutic gene; or a complement thereof.

18 The nucleic acid molecule of claim 17, wherein the therapeutic gene encodes a cytotoxic protein; or a complement thereof.

19 The nucleic acid molecule of claim 1, further comprising an internal ribosome entry site (IRES); or a complement thereof.

20 The nucleic acid molecule comprising the insert contained within the plasmid deposited with the NIAID Research and Reference Reagent Program as Accession No. ______.

21 The nucleic acid molecule comprising the insert contained within the plasmid deposited with the American Type Culture Collection as Accession No. ______.

22 An isolated nucleic acid molecule comprising a nucleic acid sequence selected from the group consisting of: SEQ ID NO:1, SEQ ID NO:2, and SEQ ID NO:3; or a complement thereof.

23 An isolated nucleic acid molecule comprising a nucleic acid sequence which is at least about 60% identical to a nucleic acid sequence selected from the group consisting of: SEQ ID NO:1, SEQ ID NO:2, and SEQ ID NO:3; or a complement thereof.

24 The nucleic acid molecule of claim 1, which is contained within a vector.

25 The nucleic acid molecule of claim 24, wherein the vector is a plasmid.

26 The nucleic acid molecule of claim 24, wherein the vector is a recombinant virus.

27 The nucleic acid molecule of claim 26, wherein the vector is a recombinant retrovirus.

28 The nucleic acid molecule of claim 27, wherein the vector is a recombinant lentivirus.

29 The nucleic acid molecule of claim 27, wherein the retrovirus is derived from HIV.

30 The nucleic acid molecule of claim 26, wherein the virus is replication incompetent.

31 A host cell containing the nucleic acid molecule of claim 1.

32 The host cell of claim 31, wherein the nucleic acid molecule is stably integrated into the genome of the cell.

33 The host cell of claim 31, which is a human T cell.

34 The host cell of any claim 33, which is a CEM T cell.

35 The host cell deposited with the NIAID Research and Reference Reagent Program as Accession No. ______.

36 The host cell deposited with the American Type Culture Collection as Accession No. ______.

37 A method of determining whether HIV is present in a sample comprising:
a) contacting the host cell of claim 31 with the sample;
b) culturing the cell for an amount of time sufficient to allow HIV infection and gene expression; and
c) determining whether the reporter gene is expressed by the cell;
wherein expression of the expressible sequence is indicative of the presence of HIV in the sample.

38 The method of claim 37, wherein the sample is a biological sample isolated from a subject.

39 The method of claim 38, wherein the subject is a human.

40 The method of claim 38, wherein the sample is selected from the group consisting of a biological fluid sample, a tissue sample, and a cell sample.

41 The method of claim 40, wherein the biological fluid is selected from the group consisting of blood, serum, plasma, saliva, urine, stool, semen, vaginal fluid, spinal fluid, lymph, amniotic fluid, tears, nasal secretions, sweat, breast milk, mucus, and interstitial fluid.

42 The method of claim 40, wherein the tissue sample is selected from the group consisting of a lymph node sample, a skin sample, and a chorionic villus sample.

43 The method of claim 40, wherein the cell sample is a blood cell sample.

44 The method of claim 43, wherein the cell sample is a T cell sample.

45 The method of claim 37, wherein the sample is purified.

46 A method of determining whether a cell is infected with HIV comprising:
a) contacting the cell with the virus of claim 26;
b) culturing the cell for an amount of time sufficient to allow HIV gene expression; and
c) determining whether the expressible sequence is expressed by the cell;
wherein expression of the expressible sequence is indicative of HIV infection of the cell.

47 The method of claim 46, wherein the cell is a T cell.

48 A method of determining whether a subject is infected with HIV comprising:
a) contacting the cells of the subject with the virus of claim 26; and
b) determining whether the expressible sequence is expressed by the cells;
wherein expression of the expressible sequence is indicative of HIV infection.

49 The method of claim 48, wherein the subject is a human.

50 The method of claim 37, wherein the step of determining whether the expressible sequence is expressed by the cell(s) comprises detecting the RNA encoded by the expressible sequence.

51 The method of claim 50, wherein the RNA is detected using a method selected from the group consisting of Northern blotting, primer extension, RT-PCR, and nuclease protection.

52 The method of claim 37, wherein the step of determining whether the expressible sequence is expressed by the cells comprises detecting the polypeptide encoded by the expressible sequence.

53 The method of claim 52, wherein the polypeptide is detected using a method selected from the group consisting of Western blotting, ELISA, and RIA.

54 The method of claim 52, wherein the polypeptide is detected using a method that detects the activity of the polypeptide.

55 The method of claim 54, wherein the method that detects the activity of the polypeptide is selected from the group consisting of a fluorescence assay, a β-galactosidase assay, a CAT assay, a luciferase assay, and a thymidine kinase assay.

56 A method of killing a cell infected with HIV comprising contacting the cell with the virus of claim 29, wherein the expressible sequence encodes a cytotoxic protein.

57 The method of claim 56, wherein the cell is a T cell.

58 The method of claim 56, wherein the cells are contained within a human subject.

59 A method of treating a subject infected with HIV comprising administering to the subject the virus of claim 26, wherein the expressible sequence encodes a cytotoxic protein.

60 A method of identifying a compound capable of inhibiting HIV infection or gene expression or comprising:
a) contacting the host cell of claim 31 with a test compound;
b) contacting the cell with HIV;
c) culturing the cell for an amount of time sufficient to allow HIV infection and gene expression; and
d) determining whether the expressible sequence is expressed by the cell,
wherein a compound that inhibits expression of the expressible sequence is identified as a compound that is capable of inhibiting HIV infection or gene expression.

61 The method of claim 60, wherein steps (a) and (b) may be performed in any order or at the same time.

62 A method of identifying a compound capable of inhibiting HIV infection or gene expression or comprising:
a) contacting a cell with HIV;
b) contacting the cell with the virus of claim 26;
c) contacting the cell with a test compound;
d) culturing the cell for an amount of time sufficient to allow HIV infection and gene expression; and
e) determining whether the expressible sequence is expressed by the cell,
wherein a compound that inhibits expression of the expressible sequence is identified as a compound that is capable of inhibiting HIV infection or gene expression.

63 The method of claim 62, wherein steps (a), (b), and (c) may be performed in any order or at the same time.

64 A method of identifying a compound capable of inhibiting HIV infection or gene expression or comprising:
a) contacting the cell infected with HIV with the retrovirus of claim 26;
b) contacting the cell with a test compound;
c) culturing the cell for an amount of time sufficient to allow HIV infection and gene expression; and
d) determining whether the expressible sequence is expressed by the cell,
wherein a compound that inhibits expression of the expressible sequence is identified as a compound that is capable of inhibiting HIV infection or gene expression.

65 The method of claim 64, wherein steps (a) and (b) may be performed in any order or at the same time.

66 The method of claim 60, wherein the step of determining whether the expressible sequence is expressed by the cell comprises detecting the RNA encoded by the reporter gene.

67 The method of claim 66, wherein the mRNA is detected using a method selected from the group consisting of Northern blotting, primer extension, RT-PCR, and nuclease protection.

68 The method of claim 60, wherein the step of determining whether the expressible sequence is expressed by the cells comprises detecting the polypeptide encoded by the reporter gene.

69 The method of claim 68, wherein the polypeptide is detected using a method selected from the group consisting of Western blotting, ELISA, and RIA.

70 The method of claim 68, wherein the polypeptide is detected using a method that detects the activity of the polypeptide.

71 The method of claim 70, wherein the method that detects the activity of the polypeptide is selected from the group consisting of a fluorescence assay, a β-galactosidase assay, a CAT assay, a luciferase assay, and a thymidine kinase assay.
